(19) United States
(12) Patent Application Publication
(10) Pub. No.: US 2006/0160127 A1
(43) Pub. Date: Jul. 20, 2006

(54) Extraction of molecules using frame

(76) Inventors: Detlev Hadbawnik, (US); Christian Wenz, (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 11/384,960

(22) Filed: Mar. 20, 2006

(30) Foreign Application Priority Data
Sep. 24, 2003 (WO) PCT/EP03/50652

(51) Int. Cl. C12Q 1/68

(52) U.S. Cl. 435/6; 204/450

(57) ABSTRACT

Disclosed is the extraction of molecules from at least one separation medium wherein a frame with different compartments is brought in contact with the separation medium, thereby dividing the separation medium into different compartments. At least one solvent is applied into the different compartments, extracting the molecules.

### BACKGROUND ART

The present invention relates to extraction of molecules from a separation medium.

A large variety of different separation techniques for molecules like nucleic acids, polypeptides or small non-polymeric organic molecules is known. All of these separation techniques, for example isoelectric focusing, sodium dodecyl sulphate polyacrylamide gel electrophoresis (SDS-PAGE), native gel electrophoresis or paper electrophoresis require the recovery of the separated molecules from the respective separation media after the separation was carried out. The recovery normally involves several different steps like detecting the molecules or molecule bands in the separation media, for example by different staining techniques, followed by manually or automated excising of the molecule bands and subsequent applying of solvent to the excised molecule bands in order to recover the molecules in a liquid phase. The molecules in the liquid phase might be further subjected to one-dimensional or multi-dimensional liquid chromatography or other separation techniques for further separation. Analysis of the separated molecules can be carried out using mass spectroscopy.

### DISCLOSURE

It is an object of the present invention to provide an improved extraction technique. This is solved by the independent claims. Favorable embodiments are subjects of further claims. Thus, embodiments of the invention provide a fast and easy to use extraction technique, which can enable to extract different molecule bands from a separation medium in one step.

According to an embodiment, extraction of molecules from at least one separation medium comprises:
A) Bringing a frame with different compartments in contact with the separation medium, thereby dividing the separation medium into different compartments,
B) Applying at least one solvent into the different compartments, extracting the molecules.

Preferred embodiments provide a fast and easy-to-handle procedure for the extraction of large amounts of different molecules, located in different areas of the at least one separation medium in one step. Due to the (preferably large) number of compartments of the frame, which divide the separation medium into different compartments with different molecules, the different molecule bands in the separation medium do not have to be processed separately anymore but can be extracted in one step by applying solvents into the different compartments. Furthermore, the different molecule bands do not have to be excised from their respective separation media anymore.

Normally, the molecules are initially separated in the separation medium and are then subjected to A). As mentioned above, different state-of-the-art separation techniques like isoelectric focusing, sodium dodecyl sulphate polyacrylamide gel electrophoresis or paper electrophoresis can be used.

In another variant, a bottomless micro-titer plate with separate compartments is used. Micro-titer plates are easy to produce and are current state-of-the-art instruments, therefore providing a cheap instrument for the separation of the separation medium into different compartments.

In a preferred embodiment, prior to A) the molecules are separated in a plurality of separation media located on a carrier. Afterwards, in A) the frame is brought in contact with the plurality of separation media, separating the plurality of separation media into different compartments.

This provides the possibility to process many separation media loaded with a large amount of molecules in one step after separation of the molecules by extracting the separated molecules in the different separation media in one step by using the frame with the different compartments. Therefore, this enables an easy extraction of molecule bands located in different separation media in one step.

It is also possible to use gel-based separation media. The gel-based separation media might be selected from polyacrylamide, agarose, dextran or starch. These separation media are useful for the separation of polypeptides, nucleic acids or other small organic molecules. Paper-based separation media can also be used, for example when a paper electrophoretic separation of molecules is carried out.

In a preferred embodiment, the at least one separation medium is furthermore subjected to motion in B). Motion during the extraction of the molecules in B) can support the extraction process by e.g. equally distributing the already extracted molecules in the liquid phase so that no local high concentrations of the molecules can occur. Therefore, motion like shaking or other means keeping the extraction medium in motion e.g. stirring or rotation might enable a continuous diffusion of the molecules from the separation medium into the solvent. It is also possible that the motion might disrupt the integrity of the separation medium, therefore simplifying the extraction of the molecules.

In another variant, in B) a voltage is applied to the at least one separation medium. The voltage also might ensure a good extraction process.

Advantageously, in A) a positioner for sealing and positioning the frame onto the at least one separation medium is applied. The positioner can e.g. comprise a force, which can be applied, pressing the frame onto the separation medium ensuring a stable positioning of the frame on the separation medium with no relative movement of the frame to the separation medium during the extraction procedure. Therefore, no slipping can occur, which might lead to mixing of molecules from different compartments.

An apparatus for separation and extraction of molecules from at least one separation medium comprises:

at least one separation medium and

a frame with compartments for separation of the separation medium into different compartments.

Such apparatus is useful for carrying out a separation of molecules e.g. polypeptides, nucleic acids or other small organic molecules using a large variety of different separation techniques like polyacrylamide gel electrophoresis, agarose gel electrophoresis or other electrophoretic separation techniques. After the separation, the molecules are normally located in different areas of the separation medium, which preferably has a strip-like form. The frame with the different compartments can then be used for separation of the different molecules in different areas of the separation medium by bringing the frame in contact with the separation medium, thereby dividing the separation medium into different compartments.

This apparatus can be especially useful for separation and extraction of large amounts of polypeptides by e.g. isoelectric focusing or polyacrylamide gel electrophoresis. The apparatus can also be used for separation of nucleic acids in an e.g. agarose gel-based separation medium. Once the position of the molecules of interest in the separation medium after the separation procedure is known, different kinds of molecules can be isolated from each other and extracted from the separation medium in a very simple and fast way.

Preferably, the apparatus comprises a plurality of strip-like separation media located on a carrier for simultaneous separation of molecules in one step. Such apparatus enables a fast and easy-to-handle procedure for separation of large amounts of molecules at the same time in a single step. Due to the frame with the different compartments, such an apparatus also allows a fast extraction procedure of the separated molecules from the plurality of strip-like separation media. The plurality of separation media can, for example, comprise polyacrylamide-based gel strips with immobilized ampholytes for isoelectric focusing procedures of polypeptides, agarose gel strips for separation of nucleic acids or polyacrylamide-based separation media for the separation of polypeptides.

Advantageously, the frame furthermore comprises a seal for sealing the different compartments. The seal can e.g. comprise flexible sealing bands e.g. rubber bands, which are present in the areas of the frame contacting the separation medium (see, for example, FIG. 4). Sealing bands or sealing rings can provide a very reliable separation of the different compartments from each other, thereby ensuring that no intermixing of the molecules from different compartments can occur during the extraction procedure.

The apparatus can furthermore comprise a positioner for positioning the frame on the separation medium. The positioner can, for example, comprise a clamp, which fixes the frame on the separation medium and prevents a slipping of the frame relative to the separation medium during the extraction procedure (see for example, FIG. 3).

In the following, embodiments will be explained in more detail with reference to the figures. All figures are just simplified schematic representations presented for illustration purposes only.

### BRIEF DESCRIPTION OF THE DRAWINGS

FIG. 1 depicts a top view of a frame, which is brought into contact with a carrier on which strip-like separation media are located.

The FIGS. 2 to 4 show cross-sectional views of a frame with different compartments located on different separation media.

FIG. 5 shows a stained isoelectric focusing gel with polypeptide fractions extracted according to one embodiment.

FIG. 6 depicts a diagram showing the distribution of the amounts of polypeptides from the fractions shown in FIG. 5 measured by the Bradford method.

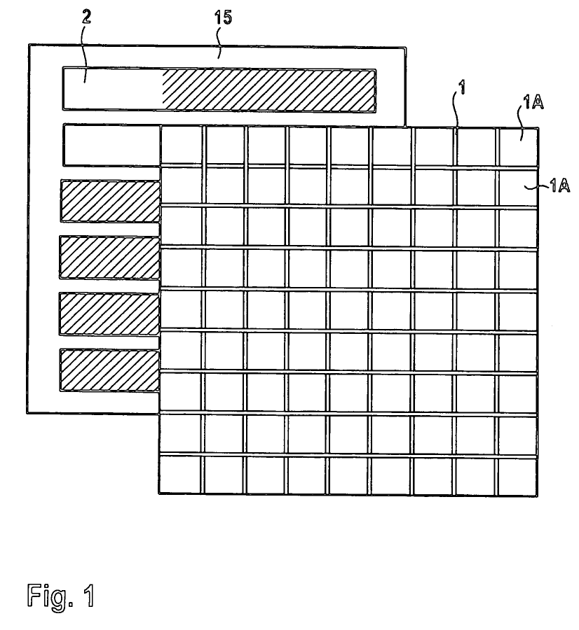

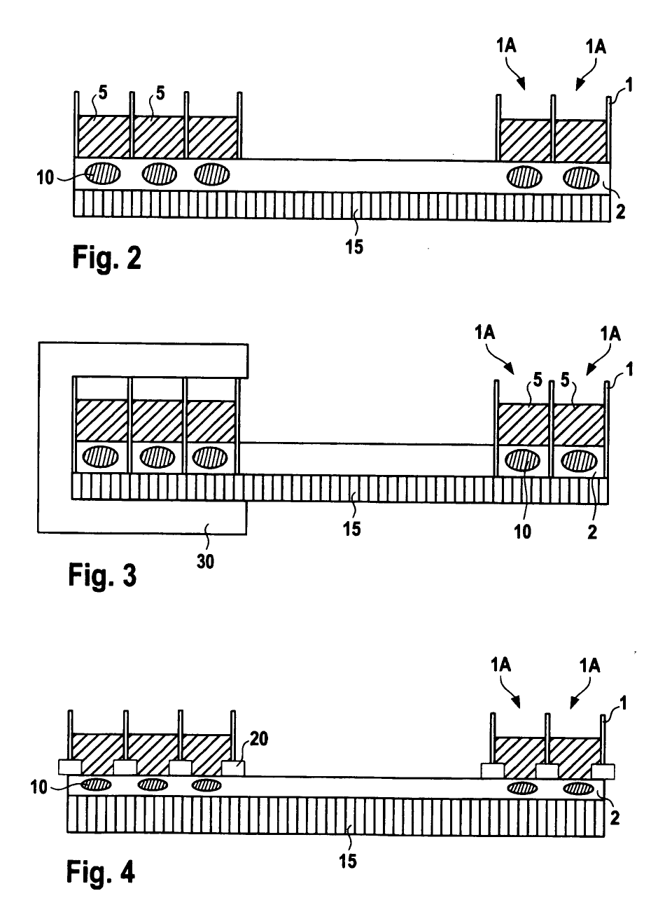

FIG. 1 shows a top view of a plurality of strip-like separation media 2 located on a carrier 15 and a frame 1 with different compartments 1A during step A) of an embodiment. The carrier 15, for example, can comprise a flat sheet made of plastic, ceramic or any other suitable carrier material. The frame 1 with the different compartments 1A is brought in contact with the plurality of separation media 2, so that each separation medium is split into different separate compartments.

FIG. 2 shows a cross-sectional view of a frame 1 and a strip-like separation medium 2 located on a carrier 15 during step B). A frame 1 with separate compartments 1A was brought in contact with the separation medium 2. The different compartments 1A preferably tightly separate different areas of the strip-like separation medium 2 in which different molecules 10 are located. Upon application of a solvent 5 into the different compartments, the different molecules 10 can easily be extracted from the separation medium 2 in one step.

FIG. 3 shows a different arrangement of a frame 1 on a strip-like separation medium 2 during step B). In this variant of step B), the frame with the different compartments 1A deeply cuts into the preferably gel-based separation medium 2, thereby cutting the separation medium into separate parts for each compartment. The frame 1 can completely cut the gel-based separation medium so that the frame 1 is in contact with the carrier 15 on which the separation medium 2 is located. It is also possible that the separation medium is just partially cut by the frame 1. A complete separation of the separation medium into different compartments as shown in FIG. 3 can ensure a complete and tight separation of the different molecules 10 located in different areas of the separation medium. Preferably, a clamp 30 for positioning the frame on the carrier 15 is present. The clamp 30 can tightly fix the frame 1 on the carrier 15, thereby preventing slipping. A clamp is especially useful when the whole arrangement of the grid, the clamp and the separation medium is subjected to shaking during step B). A solvent 5 is applied into the different compartments extracting the molecules 10 from the gel-based separation medium.

FIG. 4 again depicts another variant of the method during step B). A frame 1 having different compartments 1A is positioned on a separation medium 2. Sealing bands 20 are present in areas of the frame 1, which are in direct contact with the separation medium 2. These sealing bands 20 tightly seal the different compartments from each other preventing an intermixing of the molecules from different compartments during the extraction procedure. Sealing bands are, for example, especially useful when paper-based separation media e.g. for paper electrophoresis are used.

### Embodiment: Extraction of E. coli Proteins from Isoelectric Focusing Gels

Lyophilized Escherichia coli cells (strain B-ATCC 11303, Sigma) were suspended in buffer (7M urea, 2M thiourea, 4% CHAPS, 1% DTT) and disrupted in a BEAD-BEATER (BIOSPEC PRODUCTS) according to the recommendations of the supplier. Insoluble material was removed by centrifugation. The protein concentration of the cell extract was determined by the Bradford method and adjusted to 1 mg/ml with buffer. 0.25 ml extract were loaded on a 13 cm long Immobiline dry strip pH 4-7 (Amersham) by overnight rehydration at room temperature. Isoelectric focusing (IEF) was done using an IPGphor (Amersham) at 20° C. with a current limit of 50 μA/strip in cup loading strip holders (Amersham).

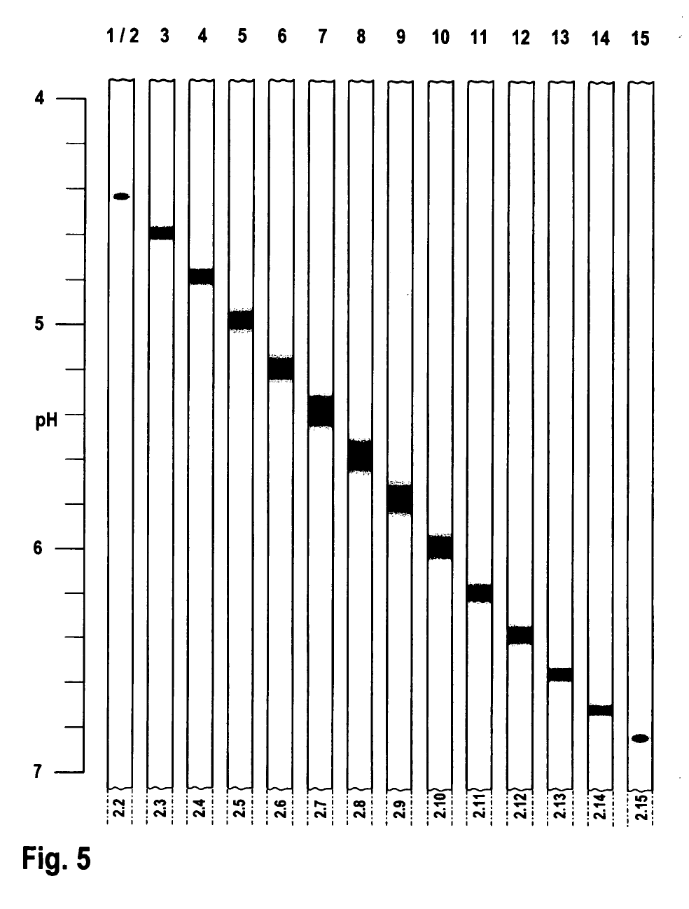

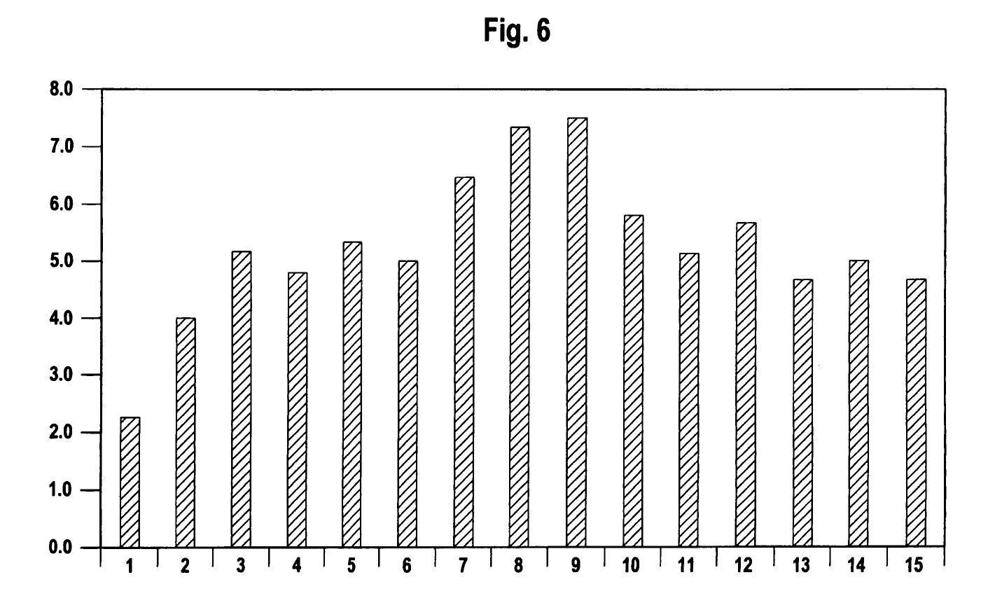

After one hour focusing at 500 V and one hour focusing at 1000 V, the voltage was set to 8000 V until a total of 20 kVh was reached. Subsequently, a frame with fifteen compartments each with a size of 0.6×0.6 mm was placed on top of the strip and 15 μl buffer was added to each compartment. The in liquid recovery was done for at least one hour with voltage (8000 V) at 20° C. or without voltage and with shaking at room temperature. The protein concentration of every fraction was measured with the Bradford method (see FIG. 6). The pl-distribution of the proteins was determined by standard one-dimensional isoelectric focusing (see FIG. 5). Every fraction was adjusted to a volume of 0.25 ml with buffer for the one-dimensional isoelectric focusing procedure. 0.5% IPG buffer pH 4-7 (Amersham) was added and the whole sample was loaded on a 13 cm long Immobiline dry strip pH 4-7 by overnight rehydration at room temperature. Conditions of the isoelectric focusing were the same as aforementioned.

FIG. 5 shows the one-dimensional isoelectric focusing gel, which was run in order to verify that the extraction of the proteins according to an embodiment of the invention did work. One can clearly see that the extraction procedure using the frame with the separate compartments resulted in an extraction of different protein bands with a resolution of about 0.1 pH units per fraction. The numbers on top of FIG. 5 mark 15 different fractions which were extracted according to the method of an embodiment of the invention from a previous isoelectric focusing gel, which was run in order to separate the proteins of the E. coli cell extract by their isoelectric points. Fractions 1 and 2 were both applied on the same gel strip. The scale on the left side of FIG. 5 shows the different pH units. The proteins were stained with PhastGel Blue R (Amersham).

FIG. 6 is a diagram showing the amount of protein in the different fractions 1 to 15 extracted from an isoelectric focusing gel. These fractions are the same as the ones shown in FIG. 5. The ordinate of FIG. 6 depicts the amount of protein in μg, recovered during step B).

The scope of the invention is not limited to the embodiments of the figures. Indeed, variations especially concerning the usage of different separation media are possible.

Embodiments of the invention can be embodied in each novel characteristic and each combination of characteristics, which includes every combination of any features, which are stated in the claims, even if this combination of features is not explicitly stated in the claims.

1. A method for extraction of molecules from at least one separation medium comprising:
A) bringing a frame with different compartments in contact with the separation medium, thereby dividing the separation medium into different compartments,
B) applying at least one solvent into the different compartments.

2. Method according to claim 1,
wherein a bottomless microtiter plate with separate compartments is used.

3. Method of claim 1,
wherein prior to step A) the molecules are separated in a plurality of separation media located on a carrier,
wherein in step A) the frame is brought in contact with the plurality of separation media, separating the plurality of separation media into different compartments.

4. Method of claim 1
wherein at least one gel-based separation medium is used.

5. Method according to claim 4,
wherein a separation medium is used, which is selected from:
polyacrylamide, agarose and dextran.

6. Method of claim 1,
wherein at least one paper-based separation medium is used.

7. Method of claim 1,
wherein in step B) the at least one separation medium is furthermore subjected to motion.

8. Method of claim 1,
wherein in step B) a voltage is applied to the at least one separation medium.

9. Method of claim 1,
wherein in A) a positioner for sealing and positioning the frame onto the at least one separation medium is applied.

10. Method of claim 1,
wherein molecules are extracted, which are selected from the following group:
nucleic acids, polypeptides and small organic molecules.

11. Apparatus for separation and extraction of molecules from at least one separation medium, comprising:
at least one separation medium and
a frame with compartments for separation of the separation medium into different compartments.

12. Apparatus of claim 11,
wherein the separation medium is a gel-based strip-like separation medium.

13. Apparatus of claim 11, further comprising:
a plurality of strip-like separation media located on a carrier for simultaneous separation of molecules in one step.

14. Apparatus of claim 11,
wherein the frame furthermore comprises a seal for sealing the different compartments.

15. Apparatus according to claim 14,
wherein flexible sealing bands are present in the areas of the frame contacting the separation medium.

16. Apparatus of claim 11, further comprising
a positioner for positioning the frame on the separation medium.

17. Apparatus according to claim 16,
wherein the positioner comprises a clamp.

18. A method for extraction of molecules from at least one separation medium comprising:
bringing a frame with different compartments in contact with the separation medium, thereby dividing the separation medium into different compartments,
applying at least one solvent into the different compartments for extracting the molecules.
